(19) United States
(12) Patent Application Publication
(10) Pub. No.: US 2021/0052292 A1
(43) Pub. Date: Feb. 25, 2021

(54) LASER SHOCKWAVE SYSTEM AND METHOD

(71) Applicant: Zlasers Ltd., Kfar Shemaryahu (IL)

(72) Inventor: Ziv Karni, Kfar Shemaryahu (IL)

(73) Assignee: Zlasers Ltd., Kfar Shemaryahu (IL)

(21) Appl. No.: 16/543,651

(22) Filed: Aug. 19, 2019

(51) Int. Cl. A61B 17/225; A61B 18/20; A61M 35/00

(52) U.S. Cl. CPC A61B 17/2251; A61B 18/203; A61B 2017/00769; A61B 2017/2253; A61M 35/00

(57) ABSTRACT

A method for application of laser energy to skin including applying a first pass of laser energy from a laser system to skin, placing a layer on the skin, and applying a second pass of laser energy from the laser system that impinges on the layer to create a shockwave on the skin.

### FIELD OF THE INVENTION

The present invention generally relates to a system and method for laser treatment of skin, and particularly to a system and method for laser energy application (such as laser pulses) combined with laser shockwave generation, which may be used, for example, in skin tightening, skin rejuvenation, or lightening or eradicating pigments in human skin, such as tattoos or pigmented skin lesions.

### BACKGROUND OF THE INVENTION

Extremely short pulse (ESP) nanosecond or picosecond laser systems can successfully lighten or eradicate a variety of pigmented lesions, such as selectively destroying tattoo pigment without causing much damage to the surrounding skin. The altered pigment is then removed from the skin by scavenging white blood cells and tissue macrophages.

Q-switching can produce light pulses with extremely short (in the range of nanoseconds) pulse duration and high (megawatt) peak power, much higher than can be produced by the same laser operating in continuous wave mode (constant output), or free-running pulse mode (0.1 ms-300 ms). Laser pulses can also be in the range of picoseconds. The ESP laser systems are effective because they confine their energy to the treated pigments. The time duration (pulse duration) of the ESP laser energy is so short that the extremely small pigments of a size of 10 nm-100 nm are heated to fragmentation temperature before their heat can dissipate to the surrounding skin. This prevents heating of the surrounding tissue that could potentially lead to burning or scarring of the skin.

The most likely cause of pigment destruction when subjecting the pigments to ESP laser pulses are shockwave and/or cavitation damage, the photomechanical physical effects produced from thermal expansion, and/or the extreme temperature gradients created within the melanosome or tattoo pigment.

However, a problem is that the laser energy can create a “whitening” condition at the treatment area that tends to reduce the effectiveness of subsequent laser exposures. The whitening is a result of the generation of vacuoles due to rapid heating or energy transfer associated with laser exposure to the tattoo or pigment particle. It is believed the vacuoles are created by localized heating from the laser light absorption by melanin in the epidermis. The dermal vacuoles associated with whitening result in the attenuation or scattering of laser light, which reduces the laser effectiveness after the initial treatment. The dermal vacuoles remain in the skin for a period of time limiting the effectiveness of subsequent laser exposures in the same session.

The whitening may fade over about twenty minutes or more following the last laser exposure. Such fading may be evidenced by the resolution of the superficial vacuoles caused by the dissipation and absorption of vacuole contents, including gas, over time.

One solution to the problem of whitening, described in U.S. patent application Ser. No. 13/753,816 (published as US 2013/0165839), is the use of perfluorodecalin (PFD) to inhibit or reduce whitening caused by laser treatment of tattoos. PFD is a liquid that is colorless and inert, has low surface tension, and is insoluble in blood and water. PFD is supposedly good at reducing whitening that is caused by vacuoles located superficially (e.g., epidermal-dermal boundary). However, due to its poor dermal penetration, vacuoles that surround and shield the previously treated intradermal pigment particles are not affected. Thus, while PFD may provide benefits in reducing the appearance of whitening, it only provides limited benefit in improving the effectiveness of repeated laser treatments to the tattoo site.

PCT Published Patent Application WO 2017/165595 discloses a method for acoustic treatment of tissue to disperse vacuoles within the tissue. Pulsed acoustic waves are directed from an acoustic wave generator into the tissue containing the vacuoles. The acoustic wave generator is a rapid pulse electrohydraulic shockwave generator, such as a megasonic wave generator configured to produce pulsed acoustic waves with a frequency between about 700 KHz and about 20 MHz.

### SUMMARY OF THE INVENTION

The present invention seeks to provide a novel and improved system and method for laser energy application (such as laser pulses) combined with laser shockwave generation, which may be used, for example, in skin tightening, skin rejuvenation, or lightening or eradicating pigments in human skin, such as tattoos or pigmented skin lesions, as described in detail below.

There is thus provided in accordance with a non-limiting embodiment of the present invention a method for application of laser energy to skin including applying a first pass of laser energy from a laser system to skin, placing a layer on the skin, and applying a second pass of laser energy from the laser system that impinges on the layer to create a shockwave on the skin. The layer may be a liquid or solid layer.

In accordance with a non-limiting embodiment of the present invention the laser system includes more than one laser source, and the first the second passes of laser energy are produced by identical laser sources of the laser system.

Alternatively, the laser system includes more than one laser source, and the first the second passes of laser energy are produced by different laser sources of the laser system.

In accordance with a non-limiting embodiment of the present invention an optical frequency of the second pass of laser energy matches a fundamental resonant frequency of the liquid layer or a harmonic thereof.

### BRIEF DESCRIPTION OF THE DRAWINGS

The present invention will be understood and appreciated more fully from the following detailed description, taken in conjunction with the drawings in which:

FIGS. 1A, 1B and 1C are simplified illustrations of a laser system, constructed and operative in accordance with a non-limiting embodiment of the present invention, respectively showing application of first laser pulses on skin of a patient, application of a liquid layer on the skin, and application of second laser pulses that in conjunction with the liquid layer generate shockwaves at the skin; and

FIGS. 2A, 2B and 2C are simplified illustrations of the laser system, constructed and operative in accordance with another non-limiting embodiment of the present invention, respectively showing application of first laser pulses on skin of a patient, application of a solid substrate on the skin, and application of second laser pulses that in conjunction with the solid substrate generate shockwaves at the skin.

### DETAILED DESCRIPTION OF EMBODIMENTS

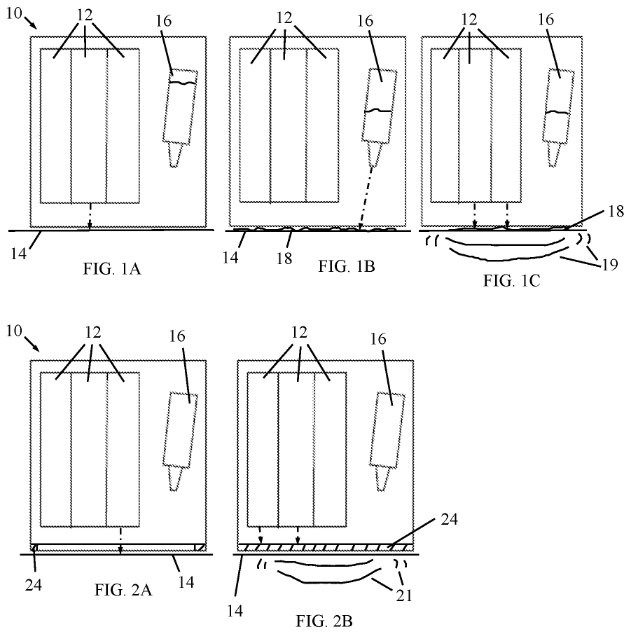

Reference is now made to FIGS. 1A-1C, which illustrate a laser system 10, constructed and operative in accordance with a non-limiting embodiment of the present invention.

System 10 includes one or more lasers 12 of different wavelengths for removing pigments of different colors. For example, laser or lasers 12 may include Q-switched Nd:YAG lasers (e.g., 532 nm frequency-doubled Nd:YAG laser), red light lasers (e.g., 694 nm ruby, 755 nm alexandrite), and near-infrared lasers (e.g., 1064 nm Nd:YAG)). The energy flux may be, without limitation, 5-10 J/cm² for a spot size of 2-4 mm.

As disclosed in PCT Patent Application PCT/IB2019/056921 to Karni, the one or more lasers 12 may cooperate with a camera and an image processor (controller), wherein the camera views the tattoo or other pigment which is to be removed. The camera may be integrated into a laser scanner. The camera senses the pigment color and the particular laser wavelength is chosen by the controller. The one or more lasers 12 are operatively coupled to the scanner and controlled by the controller that has image processing software that moves the scanner according to the contour and the color of the tattoo or pigmented area. The image processing software of the controller controls the application of the laser beams to lighten or remove the tattoo or pigment of a specific color. The camera can provide feedback to the controller regarding the change in color of the tattoo or pigment.

In one non-limiting example, as shown in FIG. 1A, the laser 12 is a Q-switched laser that emits a first laser beam application on a skin area 14, such as for a duration of 1-10 nsec, 500-1000 mJ per pulse, with 1-10 pulses per second, and a fluence of about 5-10 J/cm²).

As opposed to the prior art, in the present invention, the same laser system 10 is used to produce a shockwave. Without being bound by any theory, in general when a laser pulse of short duration (such as in the range of nanoseconds) and sufficient power (e.g., at least 100 W/cm²) is focused on the surface of a solid target, the laser energy absorption of the target surface generates a plasma whose expansion induces by reaction a shockwave. In one method, the laser energy impinging directly on the target (i.e., the surface at which the shockwave is intended to achieve a purpose, such as breaking up pigment in the skin) may cause a shockwave. However, in another method the laser energy is irradiated on a medium which is transparent to the laser energy. It is believed the transparent medium confines or retains plasma generated by the laser energy irradiated on the medium so as to greatly increase the magnitude of the shockwave (by at least one order of magnitude) and increase the shockwave duration (by a factor of two or three). The shockwave profile roughly follows the temporal characteristics of the laser pulse.

As stated in the background, it is accepted in the prior art that the most likely cause of pigment destruction when subjecting the pigments to ESP laser pulses are shockwave and/or cavitation damage, the photomechanical physical effects produced from thermal expansion, and/or the extreme temperature gradients created within the melanosome or tattoo pigment. The shockwave of the prior art is from direct irradiation of laser energy on the skin. This kind of shockwave is very limited in its ability to remove pigment; increasing direct laser irradiation causes burns or charring which is clearly unacceptable. In contradistinction to the prior art, the present invention uses a confinement layer (which may be laser-transparent, but not necessarily so), which may be liquid or solid, to produce laser-generated shockwaves, as is now described. This layer produces shockwaves without burning or charring the skin, a significant improvement over the prior art.

In one embodiment, as shown in FIG. 1B, after the first laser beam application, a liquid applicator 16 is used to apply a liquid layer 18 onto skin area 14. The liquid may be, without limitation, water, saline, and others. The liquid may also serve as a coolant to the skin area 14 to help alleviate pain. Then, as shown in FIG. 1C, one or more of the lasers 12 emits a second laser beam application on the liquid layer 18 located on the skin area 14 that produces shockwaves 19. For example, Q-switched Nd:YAG lasers have been used in lithotripsy for stone fragmentation. Optical breakdown induced by a focused Q-switched laser pulse in water can produce a shockwave of microsecond duration. The focused laser initially vaporizes the water, giving rise to a bubble that expands rapidly and generates concomitantly a divergent shock wave. Upon reaching maximum expansion, the bubble collapses violently emitting a secondary shockwave.

Other possible lasers for generating shockwaves for this purpose include, without limitation, Q-switched ruby lasers and Q-switched alexandrite lasers. In one embodiment, the optical frequency of the laser pulse matches the fundamental resonant frequency of the molecules of the liquid layer 18 or a harmonic thereof. For example, if the liquid is water, the laser optical wavelength may be about 970 nm, so as to match a principal resonant frequency of water.

The same laser that produces the first pass (before application of liquid) may be used to produce the shockwave. Alternatively, another one of the lasers in the system may produce the shockwave.

Reference is now made to FIGS. 2A-2B, which illustrate another version of laser system 10, constructed and operative in accordance with a non-limiting embodiment of the present invention.

In this version, instead of application of a liquid layer, a solid substrate 24 (also called solid layer 24) is placed on the skin 14. The high-energy, pulsed laser beam impacts substrate 24 and generates shockwaves 21. Suitable materials for substrate 24 include, without limitation, soda lime glass, plastic film (e.g., polycarbonate film, polyester film, polyethylene terephthalate (PET)d others), metals (e.g., aluminum foil). The substrate 24 may be constructed as a shutter which is open for the first laser pass and then closed for the shockwave laser pass. The laser system may include both liquid applicator 16 and substrate 24. A controller may be used to select which layer (solid or liquid or a combination thereof) to use for generating the shockwaves.

The examples described herein are for tattoo removal but the invention can be used for any skin pigment removal. For example, pigmented lesions that are treatable with the invention include, without limitation, freckles and birthmarks including congenital melanocytic nevi, blue nevi, nevi of Ota/Ito and Becker nevi.

In addition to tattoo or pigment removal, the laser system 10 may be used for other skin treatments, such as but not limited to, skin tightening, skin rejuvenation, cellulite reduction, all of which can benefit from the generation of high stress shockwaves.

What is claimed is:

1. A method for application of laser energy to skin comprising:
applying a first pass of laser energy from a laser system to skin;
placing a layer on the skin; and
applying a second pass of laser energy from said laser system that impinges on said layer to create a shockwave on the skin.

2. The method according to claim 1, wherein said layer comprises a liquid layer.

3. The method according to claim 1, wherein said layer comprises a solid layer.

4. The method according to claim 1, wherein said laser system comprises more than one laser source, and said first said second passes of laser energy are produced by identical laser sources of said laser system.

5. The method according to claim 1, wherein said laser system comprises more than one laser source, and said first said second passes of laser energy are produced by different laser sources of said laser system.

6. The method according to claim 1, wherein said first and second passes of laser energy are produced by a Q-switched laser.

7. The method according to claim 2, wherein said liquid layer is used to cool the skin.

8. The method according to claim 2, wherein said liquid layer is used to alleviate pain at the skin.

9. The method according to claim 2, wherein an optical frequency of the second pass of laser energy matches a fundamental resonant frequency of said liquid layer or a harmonic thereof.

10. The method according to claim 1, wherein said skin includes a pigmented area.
